(19) United States
(12) Patent Application Publication
(10) Pub. No.: US 2019/0298941 A1
(43) Pub. Date: Oct. 3, 2019

(54) Adjunct Diagnostic Device and Method

(71) Applicant: John R. Collins, Provo, UT (US)

(72) Inventor: John R. Collins, Provo, UT (US)

(21) Appl. No.: 16/372,975

(22) Filed: Apr. 2, 2019

Related U.S. Application Data
(60) Provisional application No. 62/651,850, filed on Apr. 3, 2018.

(51) Int. Cl. A61M 15/00; A61M 16/14; A61M 16/20

(52) U.S. Cl. CPC A61M 15/002; A61M 15/009; A61M 16/14; A61M 16/202; A61M 2205/50; A61M 2205/3331; A61M 2205/3553; A61M 2205/3584; A61M 2202/064

(57) ABSTRACT

A system for setting a medically prescribed treatment regime on a hand held inhalator device is disclosed that includes the hand held inhalator device having a transmitter/receiver configured to receive data from a medical practitioner device and send data to the medical practitioner device as well as a trigger configured to dispense medication from a medication source into a chamber of the inhalator device after a threshold level of positive pressure is achieved within the chamber and maintained for a predetermined period of time. A medical practitioner application programming interface is used to configure the predetermined period of time and the second threshold level of positive pressure of the hand held inhalator device required to actuate the trigger and dispense medication.

### PRIORITY CLAIM

The present invention claims priority to U.S. Ser. No. 62/651,850 filed on Apr. 3, 2018 entitled “Adjunct Diagnostic Device and Method” which is incorporated herein by reference in its entirety.

### FIELD OF THE INVENTION

The present invention relates to devices for delivery of medication to the airways of a patient and more particularly to improved delivery mechanisms intended to deliver medication to a patient.

### BACKGROUND

Patients who suffer from respiratory ailments including chronic obstructive pulmonary disease, asthma, bronchitis, tuberculosis, or other disorder or condition that causes respiratory distress, often self-administer medication to treat symptoms for those ailments.

Presently, many patients attempt delivery of medications to the respiratory system through hand-held metered dose inhalers (MDI) and dry powder inhalers (DPI). Small volume nebulizers (SVN) may also be used. An MDI is a device that helps deliver a specific amount of medication to the lungs, usually by supplying a short burst of aerosolized medicine that is inhaled by the patient. A typical MDI consists of a canister and an actuator (or mouthpiece). The canister itself consists of a metering dose valve with an actuating stem. The medication typically resides within the canister and is made up of the drug, a liquefied gas propellant and, in many cases, stabilizing excipient. Once assembled, the patient then uses the inhaler by pressing down on the top of the canister, with their thumb supporting the lower portion of the actuator. Actuation of the device releases a single metered dose of liquid propellant that contains the medication. Breakup of the volatile propellant into droplets, followed by rapid evaporation of these droplets, results in an aerosol consisting of micrometer-sized medication particles that are then breathed into the lungs. Other MDI's are configured to be charged by twisting a cylinder that charges the device. A button on a side of the cylinder is depressed by the user which results in a timed release of nebulized or aerosolized medication for inhalation by the patient.

DPI's involve micronized powder often packaged in single dose quantities in blisters or gel capsules containing the powdered medication to be drawn into the lungs by the user's own breath. These systems tend to be more expensive than the MDI, and patients with severely compromised lung function, such as occurs during an asthma attack, may find it difficult to generate enough airflow for satisfactory performance.

While used widely for the treatment of respiratory distress, treatment protocols using MDI's and DPI's ignore the physiological state of patients suffering from respiratory distress. That is, generally speaking, many patients presenting symptoms related to respiratory distress suffer from closed or inflamed alveoli. It is the inflammation of the airways within the lungs of the patient that causes discomfort and other symptoms related to their respiratory distress. Unfortunately, common treatment techniques related to MDI and DPI use, deliver medication to inflamed and non-inflamed airways alike. The desired physiological response to the administered medications (i.e., the opening or reduced inflammation of the airways, etc.) is delayed as the medication is absorbed into the bloodstream and thereafter delivered to the closed or inflamed airways. Moreover, use of MDI's or DPI's can be difficult to administer to very young or very old patients or others with decreased or low dexterity. For example, a patient suffering from an acute asthmatic attack may have a difficult time taking a deep enough breathe to move an aerosol from an MDI down through the patient's airway. A need exists, therefore, for improved systems and methods for lung recruitment and more efficient delivery of medication to the lung.

### BRIEF DESCRIPTION OF THE DRAWINGS

The present invention will become more fully apparent from the following description and appended claims, taken in conjunction with the accompanying drawings. Understanding that these drawings merely depict exemplary embodiments of the present invention they are, therefore, not to be considered limiting of its scope. It will be readily appreciated that the components of the present invention, as generally described and illustrated in the figures herein, could be arranged and designed in a wide variety of different configurations. Nonetheless, the invention will be described and explained with additional specificity and detail through the use of the accompanying drawings in which:

FIG. 1 is a perspective view of an inhalator device in accordance with one aspect of the technology;

FIG. 2 is a cross-section side view of the inhalator device of FIG. 1 in accordance with one aspect of the technology;

FIG. 3 is a cross-section perspective view of the inhalator device of FIG. 1 in accordance with one aspect of the technology;

FIG. 4 is a perspective view of an actuating lever of the inhalator device of FIG. 1 in accordance with one aspect of the technology;

FIG. 5 is a front view of a mouthpiece of the inhalator device of FIG. 1 in accordance with one aspect of the technology;

FIG. 6 is a front perspective view of an inhalator device in accordance with one aspect of the technology;

FIG. 7 is a cross-section side view of the inhalator device of FIG. 6;

FIG. 8 is a perspective view of an inhalator device in accordance with one aspect of the technology;

FIG. 9 is an exploded view of an inhalator device in accordance with one aspect of the technology;

FIG. 10a through 10d is a plurality of views of an inhalator device in accordance with one aspect of the technology;

FIG. 11 is a block diagram of a sensor in accordance with one aspect of the technology;

FIG. 12 is a PCB layout in accordance with one aspect of the technology;

FIG. 13 is an LED board schematic in accordance with one aspect of the technology;

FIG. 14 is an LED PCB layout in accordance with one aspect of the technology;

FIG. 15 is an inhalator command flowchart in accordance with one aspect of the technology; and

FIG. 16 is a device schematic in accordance with one aspect of the technology.

### DESCRIPTION OF ASPECTS OF THE TECHNOLOGY

Although the following detailed description contains many specifics for the purpose of illustration, a person of ordinary skill in the art will appreciate that many variations and alterations to the following details can be made and are considered to be included herein. Accordingly, the following embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

As used in this specification and the appended claims, the singular forms “a,” “an” and “the” include plural referents unless the context clearly dictates otherwise. Thus, for example, reference to “a layer” includes a plurality of such layers.

In this disclosure, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments described herein are, for example, capable of operation in other orientations than those illustrated or otherwise described herein. The term “coupled,” as used herein, is defined as directly or indirectly connected in an electrical or nonelectrical manner. Objects described herein as being “adjacent to” each other may be in physical contact with each other, in close proximity to each other, or in the same general region or area as each other, as appropriate for the context in which the phrase is used. Occurrences of the phrase “in one embodiment,” or “in one aspect,” herein do not necessarily all refer to the same embodiment or aspect.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about.” For example, for the sake of convenience and brevity, a numerical range of “about 50 angstroms to about 80 angstroms” should also be understood to provide support for the range of “50 angstroms to 80 angstroms.”

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

Reference in this specification may be made to devices, structures, systems, or methods that provide “improved” performance. It is to be understood that unless otherwise stated, such “improvement” is a measure of a benefit obtained based on a comparison to devices, structures, systems or methods in the prior art. Furthermore, it is to be understood that the degree of improved performance may vary between disclosed embodiments and that no equality or consistency in the amount, degree, or realization of improved performance is to be assumed as universally applicable.

### Example Embodiments

An initial overview of technology embodiments is provided below and specific technology embodiments are then described in further detail. This initial summary is intended to aid readers in understanding the technology more quickly, but is not intended to identify key or essential features of the technology, nor is it intended to limit the scope of the claimed subject matter, wherein the elements and features of the invention are designated by numerals throughout.

A significant part of the problem encountered in airway-related medical conditions is the reduction in airway diameter accompanying an acute attack. Bronchospasm and its attendant bronchoconstriction prevent adequate gas exchange in the lung, resulting in elevated levels of carbon dioxide and decreased levels of oxygen in arterial blood. This blood gas imbalance results in an increase in the work of breathing, which is burdensome and stressful to a patient who is often in a state of alarm. The relationship between airway caliber to pressure (or work required) to drive air from one end of a tube to another is understood. The Hagen-Poiseuille equation, also known as the Hagen-Poiseuille law, Poiseuille law or Poiseuille equation, is a physical law that describes the pressure drop in a fluid flowing through a long cylindrical pipe. It can be successfully applied to blood flow in capillaries and veins, or to air flow in lung alveoli. It is believed that the Hagen-Poiseuille equation, when applied to compressible fluids such as air, expresses pressure required to maintain volumetric flow as a function of the radius of the airway raised to the 4^{th} power. As a result, even slight changes in the radius of an airway results in a significant change in pressure required to maintain the flow of air into the lung.

With reference to asthma, as an example ailment only, the early stage of an attack is a non-homogenous process. Some airways are narrower than others, while others are effectively occluded altogether. When an aerosol, for example, is administered to the passively breathing patient, the aerosol naturally travels preferentially down the airways of greatest diameter. Certain schools of thought in aerosol administration focus primarily on particle size, quantitative deposition, and even dose metering of stimulants such as catecholamine. It is believed that the lung, if recruited to an optimal functional residual capacity (or FRC), will respond more favorably to inhaled therapy. Broadly speaking, it is believed that the optimal amount of air in a lung is present during the end of a normal expiratory phase. The volume of air may be a different percent of total lung capacity depending on the type of lung condition being treated and the methodology applied.

FRC is made of two volumes, the residual volume (RV) which is the part of the lung that never empties and expiratory reserve volume (ERV) which represents the amount of air that can be exhaled after a normal breath has been completed. FRC is generally a measure of airway and alveoli dilation which are the primary mechanisms dictating the work of breathing on a breath-to-breath basis. Narrow airways can be thought of as narrow straws which require a significant amount of pressure in order to move air. Partially open alveoli can be thought of as small balloons that have not been inflated and are small in diameter. It is difficult to get air into a lung with a low FRC. In other instances, such as a severe asthma attack, air is trapped inside a lung with a high FRC. Bronchospasm, with critically narrowed airways allowing air to enter the lung but preventing its escape results in the trapping of air in the lungs.

Lungs with both high and low FRC can be treated with appropriately applied positive end-expiratory pressure (PEEP). Put simply, applying backpressure to an air-trapped lung will allow the lung to exhale more rapidly and completely. Back pressure applied to a poorly recruited lung will allow it to move air more efficiently while the same back pressure applied to an air-trapped lung will allow it to deflate to an optimal FRC. Unfortunately, asthma attacks may occur at locations with no nearby medical facility that could administer positive end-expiratory pressure therapy to relieve suboptimal FRC and its attendant complications. Attacks could also occur near medical facilities with sub-optimal treatment options.

Aspects of the present invention relate to an improved inhalator device primarily designed to permit a patient to self-administer respiratory medication after partial recruitment of a lung or permit a medical practitioner to assist a patient to do the same. As noted above, during a respiratory attack (e.g., acute asthma, etc.) a patient's airway and alveoli can be restricted minimizing the efficient delivery of medication and causing a patient distress. It is believed that lung recruitment (i.e., opening of closed alveoli and/or restricted airways) can be achieved through positive end-expiratory pressure (PEEP) means. PEEP is used in mechanical ventilation to denote the amount of pressure above atmospheric pressure present in the airway at the end of the expiratory cycle. That is, as a patient exhales against a means designed to cause a positive back pressure against the patient's breath, it is believed that partial recruitment of the lung occurs. Thus, PEEP is believed to improve gas exchange by preventing alveolar collapse, recruiting more lung units, increasing functional residual capacity, and redistributing fluid in the alveoli.

It is intended that the inhalator devices of the present invention be operable with different types of functional attachments or components so long as the end result is partial recruitment of a patient's lung prior to dispensation of medication into the inhalator device is achieved. Bearing that in mind, the inhalator devices of the present invention, in accordance with one aspect of the invention, may be described as a hand-held housing having a mouthpiece. The mouthpiece contains apertures for allowing a patient to inhale ambient air and exhale the withdrawn air against a predetermined level of positive pressure. Within the housing, a device for detecting an amount of pressure exerted by the patient during exhalation and the time over which that pressure is exerted is present. Once a threshold level of pressure within the device has been reached over a predetermined time period, a firing mechanism triggers dispensation of medication within the housing permitting the patient to inhale the medication after partial recruitment of the lung. In one aspect of the invention, an indicator device (i.e., audible, visual, and/or tactile device) signals to the patient when a medicated breath should be taken and held. Medication is delivered via the device at the beginning of the inhaled breath to optimize the amount of medication inhaled and the depth of the medication carried down the airway. Another indicator is present providing notice to the patient that he or she may release the breath after a certain period of time.

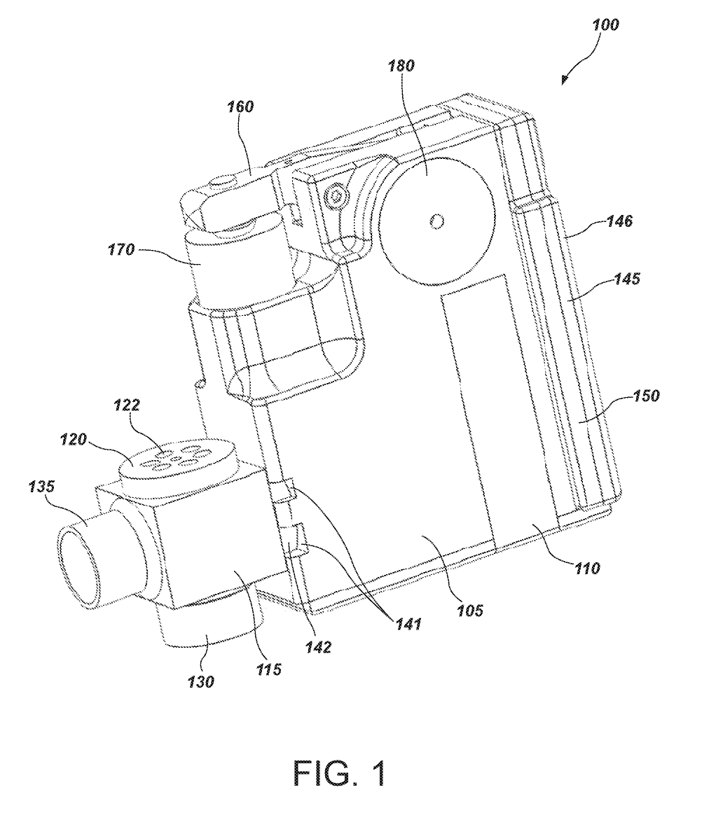

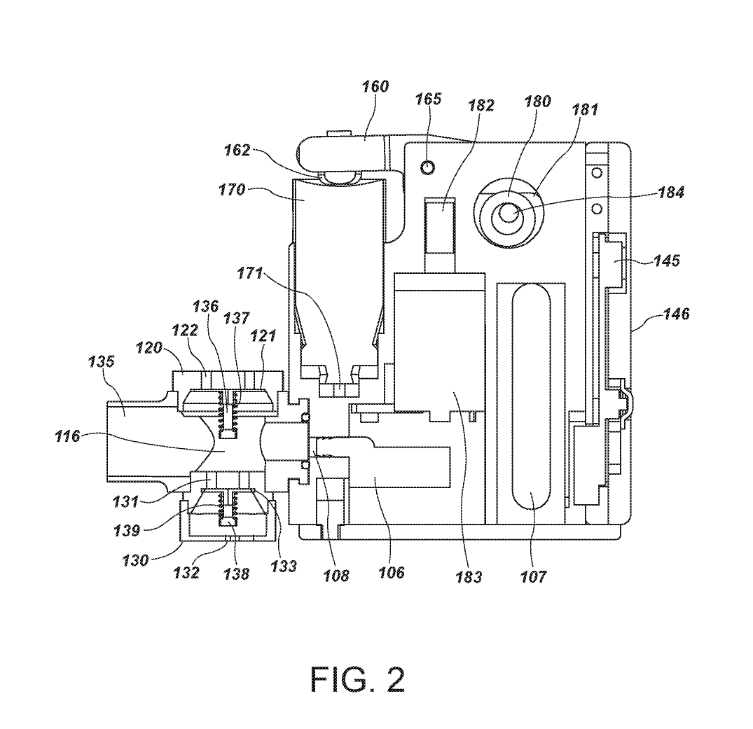

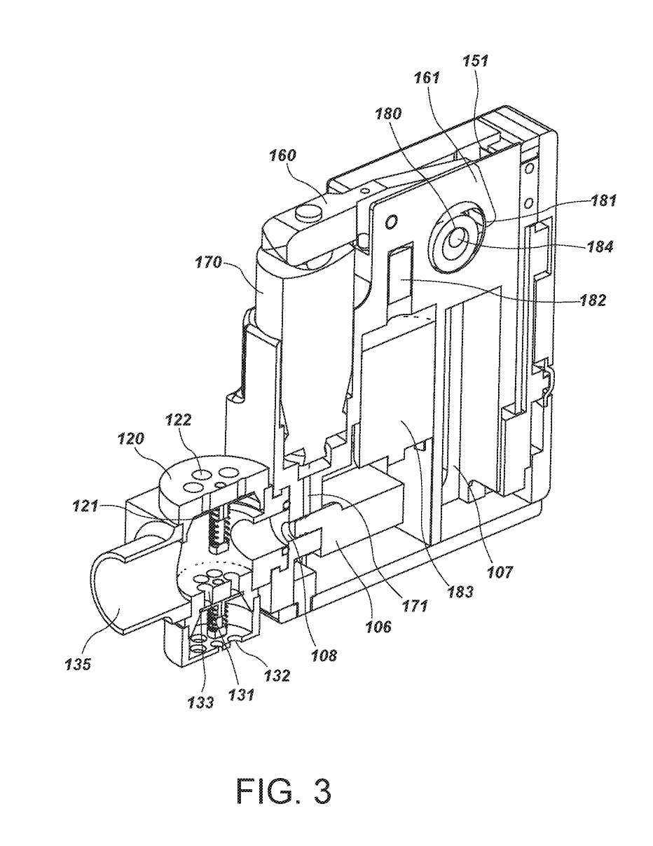

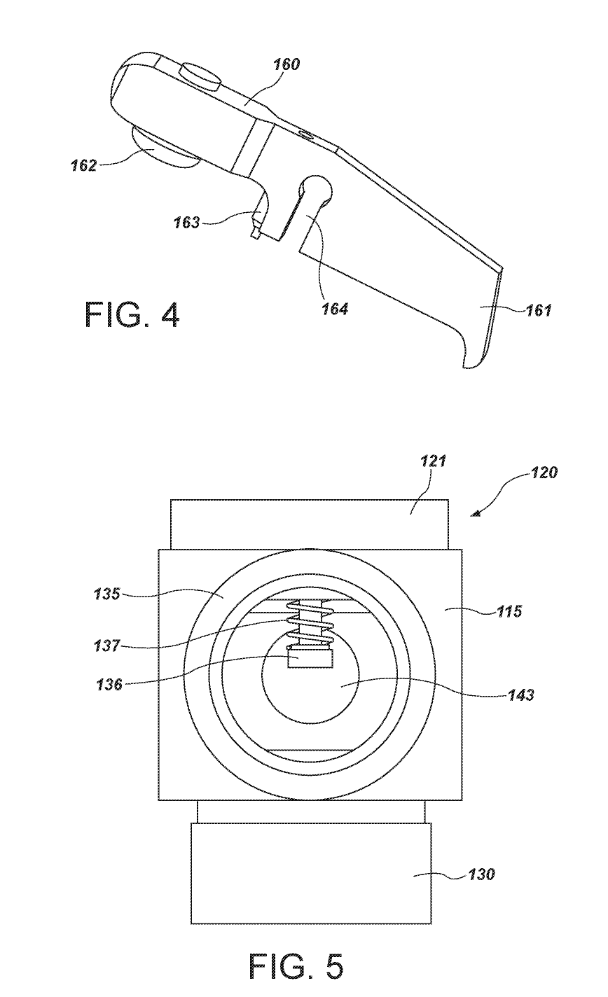

In one embodiment of the present invention, an electro-mechanical inhalator device 100 is shown. Broadly speaking, the device 100 relies on principles similar to those described above, but accomplishes the end result through use of electro-mechanical means. Referring now to FIGS. 1-5 generally, an inhalator device 100 is shown in accordance with one embodiment of the invention. The device 100 comprises an outer housing or main body 105 having a battery compartment 110. A removable mouthpiece 115 is disposed on a front end of the housing 105. A worm gear assembly 180 is disposed about a top, rear portion of the housing 105 next to an actuating lever 160. The actuating lever 160 is operatively connected to medication cartridge 170. At the rear of the device 100, a circuit board 145 is operatively connected to the device 100 for the operational sequence and trigger actuating lever 160. The circuit board base 150 is connected to the rear of housing 105.

The mouthpiece 115 comprises a primary chamber 116 with an inhale valve 120 disposed on a top portion of primary chamber 116. The inhale valve 120 comprises a plurality of apertures 122 leading from a top portion of the inhale valve 120 to a moveable plate 121. Plate 121 is disposed atop an adjustable post 136 with a spring member 137 biasing the plate 121 against the bottom of apertures 122. In this manner, the inhale valve 120 is biased in a normally closed position and is opened when negative pressure is induced within the primary chamber 116 of mouthpiece 115. In other words, the plate 121 of inhale valve 120 is moved downward when a user of the inhalator inhales sufficiently to overcome the tension of spring 137. The mouthpiece 115 also comprises a cylinder 135 configured to be inserted within the mouth of a patient. The bottom of the mouthpiece 115 comprises a valve shown generally at 130. In one aspect of the invention, though not in every aspect, the valve 130 is a PEEP valve having a plurality of inner apertures 131 on an inside of the mouthpiece 115 and atop the valve 130 and a plurality of outer apertures 132 on the outside of the mouthpiece 115 and on a bottom of the valve 130. A plate 133 is disposed atop an adjustable rod 138 and spring 139 assembly much like the inhale valve on the top of the mouthpiece 115. In contrast to the inhale valve 120, the plate 133 of the PEEP valve opens when the primary chamber 116 of the mouthpiece 115 experiences positive pressure. That is, when the user blows on the mouthpiece 115, plate 133 is directed downward against spring member 139 opening a passage between upper apertures 131 and lower apertures 132. The tension of spring member 139 may be selected in order to predetermine the quantity of pressure required to move the plate 133 downward sufficient to allow the passage of air. Both rods in the upper and lower valves may be threaded into a portion of the valve and therefore have an adjustable length. In this manner, the tension of the springs 137 and 139 may be adjusted. In one aspect of the invention, the valve 130 opens when subject to a positive pressure pre-determined by medical personnel in the range of 3 cm to 20 cm H2O and the valve 120 opens when subject to a negative pressure of not greater than 0.3 cm H2O.

The mouthpiece 115 is detachably mounted to body 105 through a plurality of grooves 141 disposed within the housing and mating lips 142 disposed within the mouthpiece 115. The grooves 141 are placed horizontally across a front face of the body 105. Mating lips 142 are likely placed horizontally across a back face of the mouthpiece 115. The mouthpiece 115 is mounted and/or removed from the body 105 by sliding the mating lips 142 horizontally through grooves 141 until the inlet 108 of the body 105 is substantially aligned with back outlet 143 of the mouthpiece 115. The groove and lip combination, however, may be arranged vertically or in an inclined plane as suits a particular design. An arrangement of circular grooves and mating lips is also contemplated for use. In this manner, the mouthpiece 115 is attached and/or detached from the body 105 of the device 100 by twisting the groove/lip mating pair into locking engagement. Other attachment means may also be used as suits a particular application and design.

A cavity is formed in the top of the body 105 configured to receive medicine cartridge 170 therein. In one aspect of the invention, medicine cartridge 170 comprises a cylindrical container with pressurized fluids therein. As with other medicine cartridges known in the art, the distal end of the cartridge comprises a stem valve 171 which, when compressed, dispenses a predetermined volume of medicine from the valve 171. The stem valve 171 is in fluid communication with inlet 108 and, once connected to the mouthpiece 115, is also in fluid communication with primary chamber 116 of mouthpiece 115.

Inlet 108 of the body 105 is in fluid communication with pressure sensor 106. When a patient blows on the mouthpiece 115, the upper valve 120 closes and the lower PEEP valve 130 opens. Depending on the tension of spring 139 and the volume of air exhaled by the patient, an amount of positive pressure within the primary chamber 116 is created. Pressure sensor 106 is configured to detect the pressure within primary chamber 116 and the amount of time pressure is continuously maintained. The pressure sensor 106 is configured to relay a signal to circuit board 145 when a qualifying breath has been achieved. Pressure sensor 106 is configured with tolerances to relay signals when a pressure that is within a predetermined (or threshold) for the predetermined (or threshold) period of time. In one embodiment the threshold pressure ranges from between 2 cm and 4 cm H2O and the threshold period of time ranges from between 2 and 6 seconds.

A qualifying breath is achieved when a patient blows through the mouthpiece 115 and creates a predetermined (or threshold) level of pressure for a predetermined (or threshold) period of time. In one aspect of the invention, the pressure sensor 106 is configured to be biased in an open or “detecting” configuration. The pressure sensor 106 closes upon detecting approximately 3 cm of H2O and re-opens upon detecting that pressure is less than 1 cm of H2O. Other pressure sensor configurations are contemplated herein as suits a particular patient's needs. In one aspect of the invention, a qualifying breath is achieved only after the patient maintains the predetermined threshold of pressure within the mouthpiece 115 for the predetermined period of time and the pressure sensor 106 detects a decrease in the pressure within the mouthpiece 115. The decrease in pressure indicates that the patient is no longer blowing into the mouthpiece 115 and is preparing to take another breath. In this manner, if the required number of qualifying breaths has been achieved, medication can be dispensed just prior to an inhalation event. Advantageously, the timing of the dispensing of the medication at the end of an exhalation cycle and just prior to an inhalation event permits the maximum inhalation of medicine into the patients lungs as medicine is drawn into the lungs at the beginning of an inhalation event (i.e., at the point of highest intake of air into the lungs). In one aspect of the invention, a qualifying breath is not achieved until after the patient maintains the predetermined threshold of pressure (e.g., between 2.8 cm and 3.2 cm of H2O) within the mouthpiece for the predetermined period of time (e.g., between 3 and 5 seconds) and the pressure sensor 106 detects a decrease in the pressure within the mouthpiece 115 to below 1 cm H2O. However, in one aspect of the invention, the pressure within the chamber on the exhalation cycle can range from between 0 and 1.5 cm H2O. Other pressures, including those on the end portion of an exhalation cycle, are contemplated herein as suits a particular application.

The pressure sensor 106 and circuit board 145 are operably connected to power source 107. In one aspect of the invention, the power source 107 is a portable power source such as a battery, rechargeable battery or the like. In yet another aspect, the entire device may be tethered to a non-portable energy source. The power source 107 and circuit board 145 are coupled to a motor 183. Once the predetermined number of qualifying breaths has been detected by the circuit board 145, the motor 183 actuates the worm 182 which in turn rotates the worm gear assembly 180. The worm gear assembly 180 comprises a worm gear and an eccentric bearing 181 disposed about a central axis 184. The worm gear assembly 180 is disposed beneath the back of actuating lever 160. When the worm assembly 180 is activated, worm 182 rotates axis 184 until the bearings 181 turn from a first position to a second position. The first bearing position is configured such that the rear 161 of the actuating lever 160 is in a downward position. The second bearing position is configured such that the rear 161 of the actuating lever 160 is in an upward position. In one aspect of the invention, the actuating lever 160 comprises a pivot pin slot 164 where the lever is mounted to the top of the housing 105. A pivot member is disposed through an aperture in the housing 105 and through the pivot pin slot 164. Actuating lever 160 also comprises an adjusting screw 162 configured to rest on top of medicine cartridge 170. When the rear 161 of actuating lever 160 is driven upward by the worm gear assembly 180, the lever 160 pivots about the pivot, driving the front of the lever 160 downward. The downward thrust of the front end of lever 160 drives the medicine cartridge 170 downward and actuates stem valve 171 releasing a dose of medicine.

A return spring cartridge 163 is disposed beneath the lever 160 near the pivot slot 164. The return spring cartridge 163 is configured to bias the rear of the lever 160 in a downward position. In this manner, after the worm gear assembly 180 drives the rear 161 of the actuating lever 160 upward, the return spring cartridge 163 will push the rear end 161 back down to compensate for a slow return of the medication cartridge 170 return action. The actuating lever 160 is designed such that the rear 161 of the actuating lever 160 comes into contact with switch 151 after stem valve 171 is actuated. When actuated, switch 151 closes a circuit sending a current to the motor 183 (thereby operating the worm assembly 180) until the lever 160 returns to a position where switch 151 is disengaged (i.e., lever is in a downward position). This terminates the circuit and its attendant current to the motor 183 ending operation of the worm gear assembly 180. In this manner, the worm gear assembly 180 and lever 160 are returned to a “pre-firing” state readying the device 100 for its next use.

Circuit board 145 is covered by a board cover 146 and is mounted to a base 150. The circuit board 145 is a printed circuit board, or PCB, used to mechanically support and electrically connect electronic components using conductive pathways, tracks or signal traces etched from copper sheets laminated onto a non-conductive substrate, but may comprise any circuit board known in the art capable of carrying out the logic described herein. In one aspect of the invention, the circuit board comprises a PLC circuit or programmable logic controller circuit. A PLC may include a sequential relay control, motion control, process control, distributed control systems, and/or networking as is known in the art. In other aspects of the invention, PLRs (programmable logic relays) may be used. PLR products such as PICO Controller, NANO PLC, and others known in the art are contemplated for use herein. In one aspect of the invention, the circuit board 145 has a memory storage component capable of storing information related to the number of times the device has been fired as the result of the user having achieved the required number of qualifying breaths. In one aspect of the invention, the circuit board 145 includes a data port which may be operably connected to a computer terminal. In this manner, the circuit board logic may be programmed to adjust the number of qualifying breaths required to actuate the actuation lever 160. A computer readable software program capable of operating on any computer operating system known in the art is configured to communicate with the circuit board 145 via a physical connection with the computer system. However, the data may also be relayed to the computer operating system via a wireless signal.

A plurality of LED's are mounted to the circuit board 145 and aligned along an edge of the housing 105 of the device 100 to be visible through the mounting base 150. In one aspect of the invention, the lights all turn on when a user picks up the inhalator 100 or creates a minimum amount of pressure within the primary chamber 116 via an initializing breath. For each qualifying breath thereafter, one of the plurality of lights is extinguished. When the last light is extinguished a green light appears indicating to the user that medication is going to be administered and that the patient should inhale the medication and hold the breath until the green light turns off. In one aspect of the invention, the appearance of the green light is coincident to the actuation of lever 160. In an additional aspect of the invention, the patient hears an audible tone also indicating that medication is going to be administered and that the patient should inhale the medicine. The timing and sequence of the lighting and/or sound, however, are adjustable as suits a particular application. For example, a single yellow light can appear for each qualifying breath leading to a final green light. In other words, for each exhalation event that reaches the predetermined pressure for the predetermined quantity of time, a yellow light appears. Once the required number of yellow lights is established, a green light appears and medication is administered. The sequence and timing are adjustable via a connection to a computer terminal or PLC controls or individual control switches mounted directly to the circuit board 145.

Other sequences or visual and/or audible indicators of the administration of medication are contemplated for use herein. For example, in one embodiment of the invention the pressure sensor 106 is configured to transmit a signal to the circuit board 145 when a first threshold of pressure is detected and when a subsequent lower threshold of pressure is detected. In this manner, an inference may be made generally when the user has ceased blowing on the inhalator 100. The first threshold pressure (i.e., for transmitting the signal) may be from 3 cm to 10 cm H2O and the second lower threshold pressure (i.e., indicating a breath has terminated) may be from 0.5 cm to 1 cm H2O, though other pressure ranges may be used. In one aspect of the invention, the motor 183 will not actuate the worm gear assembly 180 and subsequently administer medication to the patient until after the predetermined number of qualifying breaths has been achieved and after the user has ceased blowing on the inhalator 100.

In yet another aspect of the invention, a tactile sensor is placed on the cylinder 135 of mouthpiece 115. The tactile sensor is operably connected to the circuit board 145 and is designed to send a signal to the circuit board 145 when placed into contact with the skin of a patient. In one embodiment, the circuit board 145 is configured to place the inhalator 100 into “sleep mode” to preserve battery power until the tactile sensor is actuated. In another embodiment, the circuit board 145 is configured to provide an audible, visual, and/or tactile signal to the user as a reminder that the user should keep his or her mouth on the cylinder 135 during the entire exhalation and inhalation process. In other words, once the tactile sensor is actuated, a signal is provided to the user if contact with the tactile sensor is terminated prior to the actuation of the firing piston. In yet another embodiment, if contact with the tactile sensor is terminated prior to actuation of the firing piston, the circuit board 145 is configured to prevent actuation of the piston despite having detected the predetermined number of qualifying breaths. In this manner, medication will only be discharged if the number of qualifying breaths has been achieved, the user has ceased blowing on the device 100, and contact between the skin of the user and the mouthpiece 115 is maintained.

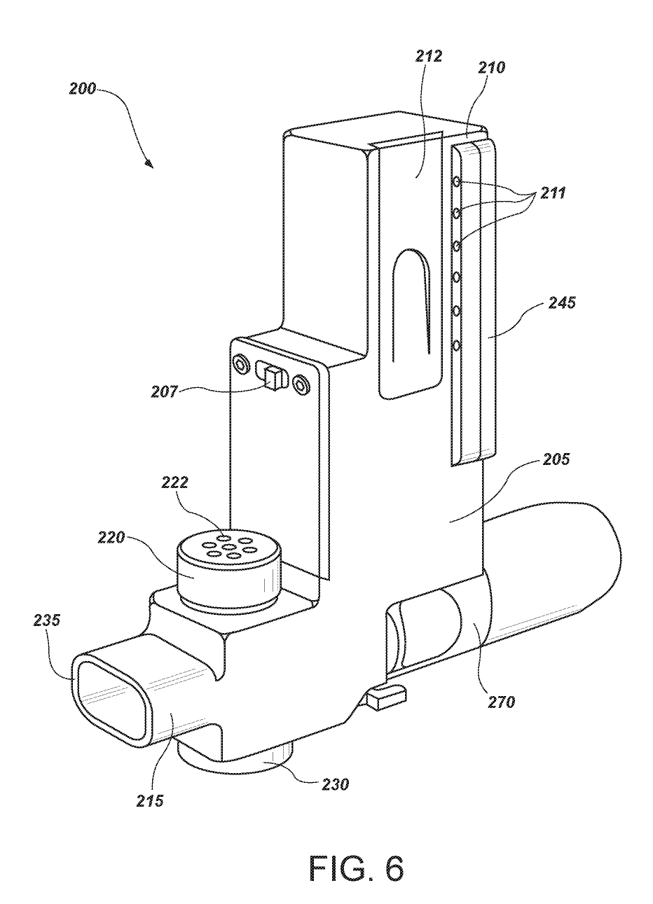

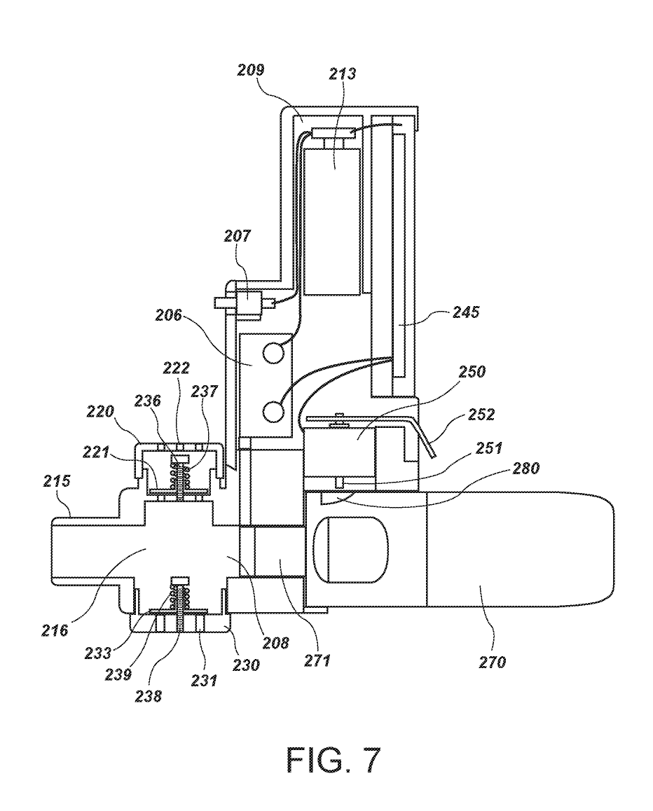

With reference now to FIGS. 6-7, in accordance with one aspect of the invention, an inhalator device 200 is shown. Similar to the inhalator device 100, this device comprises a mouthpiece 215 having a primary chamber 216 with an inhale valve 230 disposed on a bottom portion of primary chamber 216. The inhale valve 230 comprises a plurality of apertures 231 leading from a bottom portion of the inhale valve 230 to a moveable plate 233. Plate 233 is disposed below an adjustable post 238 with a spring member 239 biasing the plate 233 against the top of apertures 231. The inhale valve 230 is biased in a normally closed position and is opened when negative pressure is induced within the primary chamber 216 of mouthpiece 215. In other words, the plate 233 of inhale valve 230 is moved upward when a user of the inhalator 200 inhales sufficiently to overcome the tension of spring 239 opening an airway permitting the ingress of air into the mouthpiece 215. The mouthpiece 215 comprises an oval 235 configured to be inserted into the mouth of a patient. The top of the mouthpiece 215 comprises a valve shown generally at 220. In one aspect of the invention, the valve 220 comprises a PEEP valve having a plurality of apertures 222 on the outside of the mouthpiece 215 and on a top of the valve 220. A plate 221 is disposed below an adjustable rod 236 and spring 237 assembly much like the inhale valve 230 on the bottom of the mouthpiece 215. In contrast to the inhale valve 230, the plate 221 of the PEEP valve 220 opens when the primary chamber 216 of the mouthpiece 215 experiences positive pressure. That is, when the user blows on the mouthpiece 215, plate 221 is directed upward against spring member 237 opening a passage between apertures 222 and the ambient air. The tension of spring member 237 may be selected in order to predetermine the quantity of pressure required to move the plate 221 upward sufficient to allow the passage of air. Both rods in the upper and lower valves may be threaded into a portion of the valve and therefore have an adjustable length. In this manner, the tension of the springs 237 and 239 may be adjusted.

A cavity is formed in the back of the housing 205 configured to receive a medicine cartridge 270 therein. In one aspect, medicine cartridge 270 comprises a cylindrical container with pressurized fluids therein. The distal end of the cartridge 270 comprises a valve 271. The valve 271 is operatively coupled to a button 280 on the side of the cartridge 270. When the device 200 is charged, a predetermined volume of medicine is disposed from the valve 271 when the button 280 is depressed. The valve 271 is in fluid communication with inlet 208 and, once connected to the mouthpiece 215, is also in fluid communication with primary chamber 216 of mouthpiece 215.

Inlet 208 of the housing 205 is also in fluid communication with pressure sensor 206. When a patient blows on the mouthpiece 215, the lower valve 230 closes and the upper PEEP valve 220 opens. Depending on the tension of spring 237 and the volume of air exhaled by the patient, an amount of positive pressure within the primary chamber 216 is created. Pressure sensor 206 is configured to detect the pressure within primary chamber 216 and the amount of time pressure is continuously maintained. The pressure sensor 206 is configured to relay a signal to circuit board 245 when a qualifying breath has been achieved. Pressure sensor 206 is configured with tolerances to relay signals when a pressure that falls within a predetermined range for the pre-determined period of time similar to those ranges discussed herein. The circuit board 245 is operatively coupled to motor 250. Motor 250 is positioned such that when the cartridge 270 is properly disposed within the rear of housing 205, a piston 251 disposed about the bottom of the motor 250 is positioned directly above the button 280. When activated, motor 250 drives piston 251 downward to dispense the medication.

A bypass trigger 252 is disposed on the back of the housing 205. The bypass trigger 252 is operatively coupled to piston 251 which activates the button 280. In this manner, in the event the device 200 does not fire as anticipated, or the patient is not capable of creating the prescribed pressure within the device 200 for the predetermined number of breaths or the predetermined amount of time, the patient may manually fire the device 200 by depressing the trigger 252 and administer medication. In one aspect of the invention, the housing 205 comprises a battery 206 operatively coupled to an on/off switch 207 and the circuit board 245. The housing 205 comprises a removable plate 208 accessing compartment 209 that contains the battery 206. A plurality of lights 211 are disposed on the side 210 of housing 205. As noted above, in one aspect of the invention, lights may be activated in any number of sequences to indicate that a qualifying breath has been achieved, that medication is being administered and an inhalation breath should be taken and held, and/or how long an inhalation breath should be held.

The devices and embodiments shown herein make reference to valves for inhalation and valves for exhalation. However, in one aspect of the invention only one valve is present restricting the exhalation flow out of the mouth of the patient through the chamber. In yet another embodiment, a two-way valve may be used that provides means for the ingress of ambient air into the chamber for patient inhalation and also provides means for restricting the exhalation flow out of the mouth of the patient. In another embodiment, the chamber does not have any valves. Rather, a volume of exhalation flow from the patient is restricted by placing a plurality of holes about the exterior of the mouthpiece or other location in the housing of the device in fluid communication with the mouthpiece. Like the embodiments described above, the amount of pressure required to activate the valve is adjustable as suits a particular application by valve design and/or sizing and number of holes placed in the mouthpiece.

A method of administering medication to a patient comprises providing a hand-held, portable inhalator device to the patient, the device comprising a mouthpiece comprising a chamber and a medication source in fluid communication with the chamber. The mouthpiece further comprises an aperture configured to permit egress of fluid out of the chamber. The device also comprises a trigger configured to dispense medication from the medication source into the chamber. The method further comprises placing the mouth of the patient about the mouthpiece and exhaling into the mouthpiece and out of the aperture for a predetermined period of time at a threshold level of positive pressure to achieve a qualifying breath and dispensing a quantity of medication into the chamber after the qualifying breath. In one aspect of the invention, the method further comprises dispensing the quantity of medication into the chamber after a plurality of qualifying breaths as suits a particular prescription or patient need. In another aspect, each qualifying breath comprises exhaling through the mouthpiece for between approximately 3 and 5 seconds at a pressure within the mouthpiece ranging from between approximately 2.8 cm to 3.2 cm H2O and the patient is provided with a visual or audible indicator when a qualifying breath has been achieved. In another aspect of the invention, contact between the mouth of the patient and the mouthpiece of the device is substantially constant between qualifying breaths.

In another aspect, a method of administering medication to a patient comprises placing an inhalator device into the mouth of a patient. The device comprises a mouthpiece comprising a chamber, a fluid outlet, and a fluid inlet. It also comprises a medication source in fluid communication with the chamber and a first valve disposed about the fluid inlet. The first valve is biased in a closed position and configured to open to permit the ingress of ambient air into the chamber when subject to a threshold level of negative pressure. A second valve is disposed about the fluid outlet and is biased in a closed position and configured to open when subject to a first threshold positive expiratory end pressure to permit egress of fluid from the chamber. A trigger is disposed on the device and configured to dispense medication into the chamber. The method further comprises exhaling through the mouthpiece for a threshold period of time at a second threshold level of positive pressure and dispensing a quantity of medication into the chamber after the second threshold level of positive pressure is maintained within the chamber for a threshold period of time.

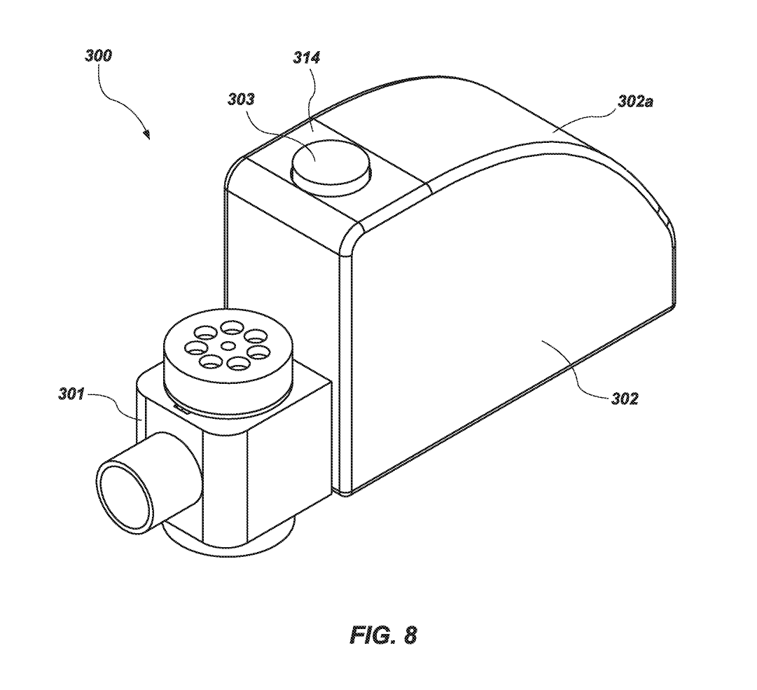

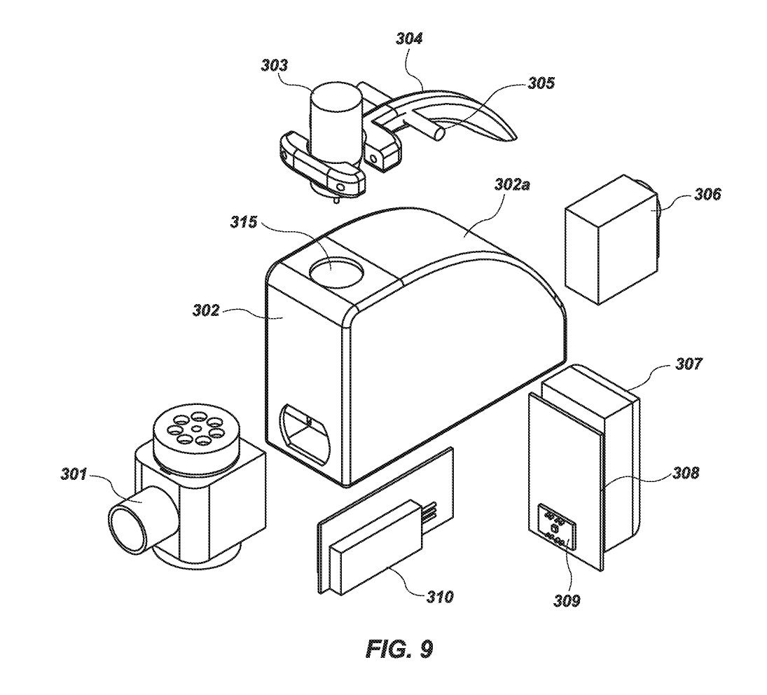

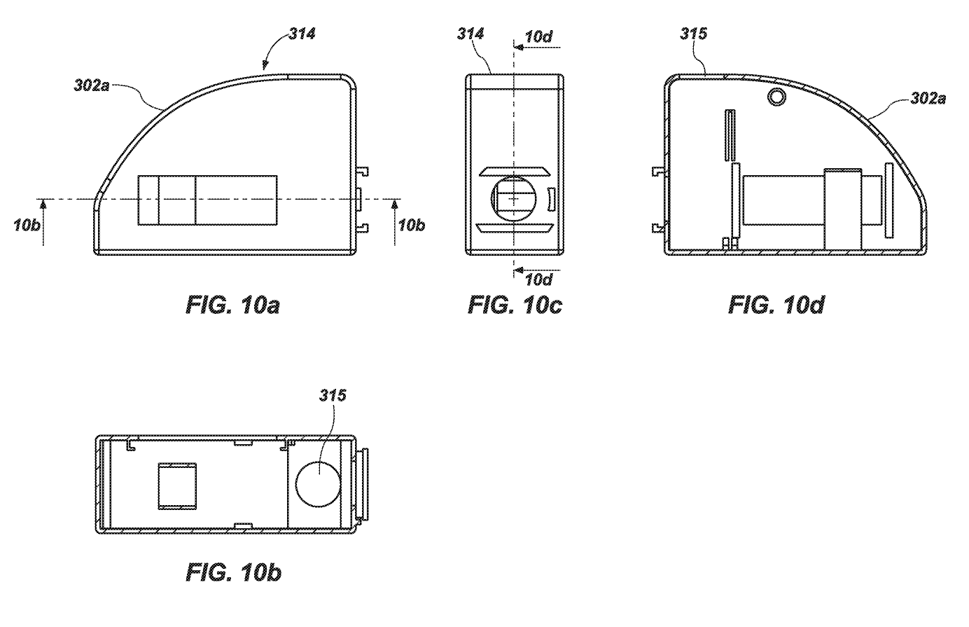

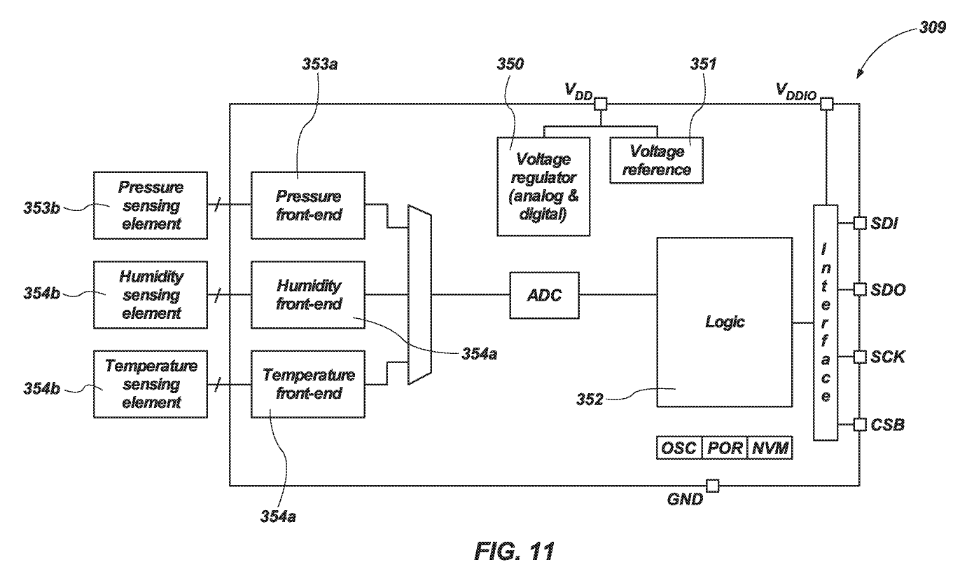

Referring generally to FIGS. 8 through 11, in another aspect of the technology, an untethered handheld inhalator device 300 is disclosed. The inhalator device generally comprises a mouthpiece 301 coupled to a housing 302. The mouthpiece comprises a plurality of valves configured to permit the patient to recruit his/her lung capacity as described above, though the valves could be disposed about other portions of the housing 302. Meaning, the valves are shown on the mouthpiece in the drawings, but that is not a requirement of the technology. Rather, the valves need only function in connection with a pressure sensor within a chamber of the device 300 to detect the “qualifying breath” programmed into the device. An inner chamber of the housing 302 is sealed such that air flow in and out of the housing 302 is regulated by the valves and openings of the mouthpiece and/or the housing 302. The inner chamber of the housing 302 is coupled to a pressure sensor 309 that cooperates with the other components described below to provide operational parameters for the actuation of a pressurized canister 303. The housing 302 is coupled to the pressurized canister 303 containing medication to be delivered to the patient. In one aspect of the technology, the pressurized canister 303 is a metered-dose-inhaler that is actuated by moving a tip of the canister 303 inward thereby releasing a predetermined quantity of pressurized medicine from the canister 303 out the tip. A top of the pressurized canister 303 is placed through an aperture in the top of the housing 302 to permit a patient to manually operate the device 300 if necessary. The canister 303 is also operatively coupled to a lever arm 304. The lever arm 304 is equipped with a pivot arm 305 that is functional in cooperation with gear 306 to automatically pivot the lever arm 304 about the pivot arm 305. In this manner, the tip of the canister 303 is moved downward against a fixed base which actuates the canister 303. A back end of the lever arm 304 is curved to approximate the curve of the back side of the housing 302. The gear 306 is operatively coupled to power source 307 which powers the gear 306 as well as the primary printed circuit board (PCB) 308, the pressure sensor 309, and display 310. The primary PCB 308 is coupled to a logic control circuit and the pressure sensor 309 comprises one or more break out circuits for optimal operation. With reference to FIG. 11, in accordance with one aspect of the technology, the pressure sensor 309 comprises PCB having a voltage regulator 350 coupled to a voltage reference 351. A pressure front end 353a is coupled to a pressure sensing element 353b. A humidity front end 354a is coupled to a humidity sensing element 354b. A temperature front end 354a is coupled to a temperature sensing element 354b. The sensing elements are all coupled to a logic circuit 352. While a pressure sensor 309 is specifically referenced herein, the technology is not limited to a sensor that detects merely pressure. For example, a pressure transducer can be used as a pressure sensor 309, but any sensor that may be used to calculate pressure or a change in pressure is contemplated for use herein. This includes, but is not limited to, ultrasonic devices, piezoelectric devices, resonant frequency devices, optical fiber sensors, and the like.

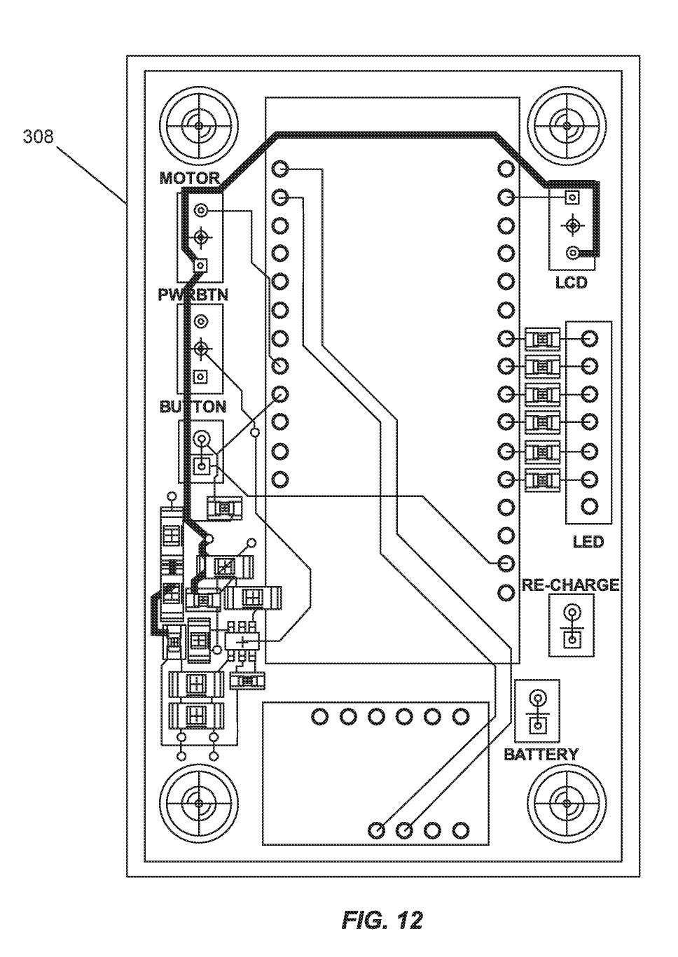

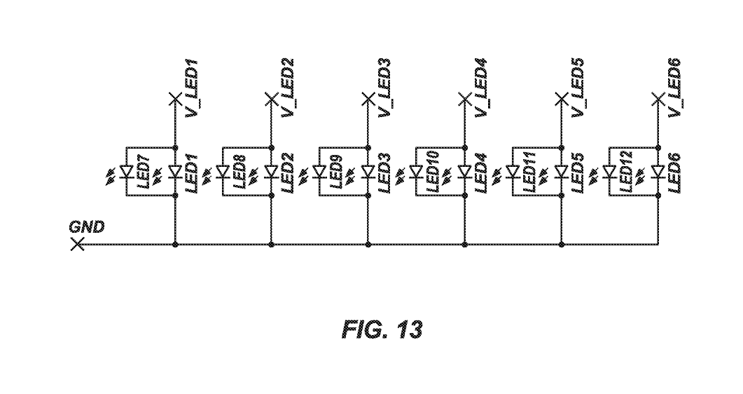

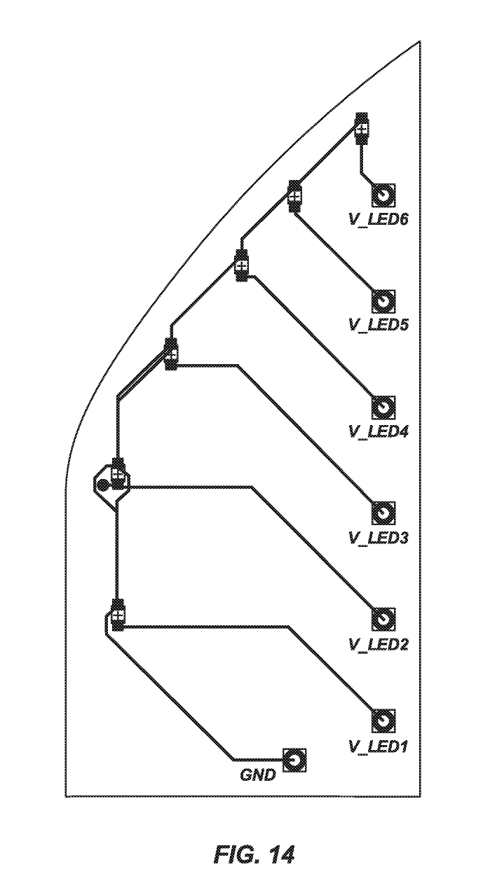

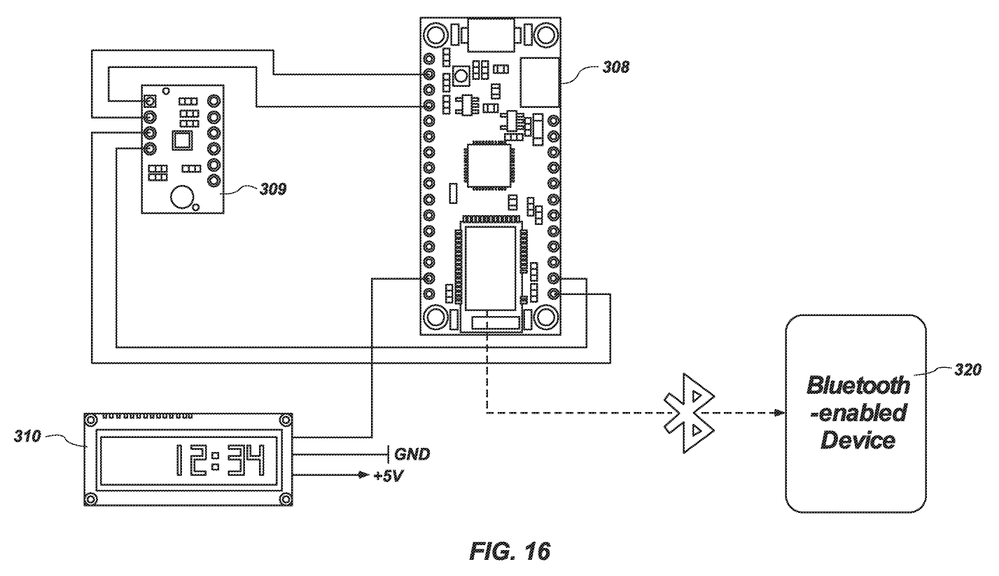

With reference generally to FIGS. 12-16, in one aspect of the technology, the logic control circuit coupled to the PCB 308 comprises an Adafruit Feather 32u4 Bluefruit LE board, though other control circuits may be used. The control circuit is coupled to the pressure sensor 309 and its break out board, and two additional PCBs. The PCB 308 provides voltage regulation from the power source 307 at 7.4 V down to 5 V. In one aspect of the technology, the control circuit and pressure sensor break out boards are soldered to the Main PCB 308 via male pin headers. The logic circuit communicates with the pressure sensor via I2C communication, and uses the signal generated from the pressure sensor to count “qualified breaths” and measure peak flow through outlet valves in the mouthpiece. The logic control circuit communicates serially to the display 310 (such as an OLED or LED), and via pre-established servo libraries to the motor 306. A second PCB is used as a peripheral to hold LEDs in a desired position on the device to display “qualified breaths,” and a countdown for a peak flow test. The PCB 308 is coupled to a plurality of LEDs (e.g., shown in FIGS. 13 and 14) that are mounted on an exterior of the device 300 or are visible through an aperture or transparent portion of housing 302. The LEDs function as described elsewhere in this application to provide, for example, visible indicators of functionality of the device 300, operational prompts, or as an indicator of the user's measured expiratory flow. In FIG. 13, the parallel LEDS are one aspect showing a layout of a mirrored PCB with LEDs mounted thereon. Generally speaking, however, either LEDs 1-6 or LEDs 7-12 would be mounted on a single board (see, e.g., FIG. 14).

In one aspect of the technology, the logic circuit is programmed using Arduino IDE (though other languages known to one in the art may be used), and implements system controls. In one aspect of the technology, a mode-select button is located on the PCB 308. Based on the position of a mode-select button (which is read from a digital i/o pin), the device 300 is placed in a peak flow cycle (i.e., peak expiratory flow rate (PEFR) cycle) or an actuation cycle. When the device 300 is in a peak flow cycle, the sensor 309 detects base pressure, temperature, and humidity within the housing 302 and altitude of the device 300 generally. When those parameters are measured and the sensor 309 detects an increase in pressure, the sensor 309 initiates a countdown and samples pressure for, for example, 4 seconds. The pressure measurement is converted into a flowrate. From these samples, the sensor 309 calculates the maximum flow (or peak flow rate), and stores that along with the base temperature, humidity, and altitude measurements and stores the measurements in a memory component of the sensor 309, PCB 308, or other storage location (e.g., other memory storage devices on device 300 or at a remote location such as a cloud-based storage).

In one aspect of the technology, the memory component comprises a non-volatile memory such as electrically erasable programmable read-only memory (EEPROM). Non-volatile memory is a type of computer memory that can retrieve stored information even after power has been cycled (turned off and back on). Examples of non-volatile memory include read-only memory, flash memory, ferroelectric RAM, most types of magnetic computer storage devices. In another aspect, however, the memory component comprises volatile memory. In addition to storing the sensor measurements, data from the sensor 309 is displayed on display 310 which may be located in an aperture through the side of housing 302. Alternatively, the display 310 may be placed about a window in the housing 302.

In one aspect of the technology, the logic control board and/or PCB 308 and/or the sensor 309 are bluetooth capable. Meaning, the inhalator device 300 contains a radio transmitter and receiver. In this manner, data measurements can be retrieved from EEPROM via a bluetooth connection from a phone or other bluetooth-enabled device 320 (iPad, PC, tablet, mobile phone, etc.) as desired. The data measurements can then be used by a medical practitioner for diagnosis, evaluation, and/or treatment of the patient. The PCB 308 can be programmed such that the amount of pressure required to generate a “qualified” breath may be tailored to the individual patient. Thus, a medical practitioner may prescribe a specific operational protocol for each individual patient based on their medical history and/or unique needs. For example, an asthmatic child may have to generate a lesser amount of pressure to create a “qualified breath” for proper lung recruitment than an 80 year old patient suffering from emphysema and thus the device 300 is programmed such that the specific response from the device 300 is unique to the patient. Both patients may also have a different number of “qualified breaths” that are required before a prescribed dose of medicine may be administered. Programming the PCB 308 to unique patient needs creates a customized medical device that can be a “prescribed device.” In one aspect, the PCB 308 can be programmed remotely through the blue-tooth connection (or other remote connection) for the unique prescription of any user.

In one aspect of the technology, the bluetooth connection provides for actuation and/or programming of the device 300 from the bluetooth enabled device 320. For example, in one aspect of the technology, the bluetooth-enable device 320 comprises an application programming interface (API) that is programmed to retrieve and process information from the device that is specifically correlated to the patient. When prescribing a dose regimen for a patient, the medical practitioner may couple his/her bluetooth enabled device (i.e., the practitioner communication device) 320 to the programmable inhaler 300. The practitioner API interfaces with the PCB 308 (or other programmable components) and allows the medical practitioner to set the pressure limits, maximum air flow, and/or total number of qualified breaths required for the device to auto-actuate and administer medicine to the patient. While a bluetooth connection is specifically referenced, it is understood that any other wireless or wired connection to the device 300 is contemplated herein that will allow the practitioner to configure the settings of the device 300 for the individual patient needs. For example, RF communication protocol, an infrared communication protocol, a wireless USB communication protocol, a ZigBee communication protocol, a cellular communication protocol, a Wi-Fi (IEEE 802.11x) communication protocol, or an equivalent wireless communication protocol which would allow secure, wireless communication of several units (for example, per HIPPA requirements) while avoiding potential data collision and interference, could be used. In another aspect, however, the device 300 can be configured manually without a wired or wireless connection to a peripheral device.

Any number of different API configurations are contemplated for use herein so long as an enable device 320 (or other communication device coupled to the device 300) allows for remote monitoring and “prescription” of the inhalator 300. Generally speaking, an API is related, or refers to, a software library. The API describes and prescribes a specification or set of rules while the library is an actual implementation of this set of rules. A single API can have multiple implementations in the form of different libraries that share the same programming interface. The separation of the API from its implementation can allow programs written in one language to use a library written in another. For example, because Scala and Java compile to compatible bytecode, Scala developers can take advantage of any Java API. API use can vary depending on the type of programming language involved. An API for a procedural language such as Lua could consist primarily of basic routines to execute code, manipulate data or handle errors while an API for an object-oriented language, such as Java, would provide a specification of classes and its class methods. Language bindings are also APIs. By mapping the features and capabilities of one language to an interface implemented in another language, a language binding allows a library or service written in one language to be used when developing in another language. Tools such as SWIG and F2PY, a Fortran-to-Python interface generator, facilitate the creation of such interfaces. An API can also be related to a software framework. A framework can be based on several libraries implementing several APIs, but unlike the normal use of an API, the access to the behavior built into the framework is mediated by extending its content with new classes plugged into the framework itself. Moreover, the overall program flow of control can be out of the control of the caller and in the hands of the framework by inversion of control or a similar mechanism.

Remote APIs allow developers to manipulate remote resources through protocols, specific standards for communication that allow different technologies to work together, regardless of language or platform. For example, the Java Database Connectivity API allows developers to query many different types of databases with the same set of functions, while the Java remote method invocation API uses the Java Remote Method Protocol to allow invocation of functions that operate remotely, but appear local to the developer. Web APIs are the defined interfaces through which interactions happen between an enterprise and applications that use its assets, which also is a Service Level Agreement (SLA) to specify the functional provider and expose the service path or URL for its API users. When used in the context of web development, an API is typically defined as a set of specifications, such as Hypertext Transfer Protocol (HTTP) request messages, along with a definition of the structure of response messages, usually in an Extensible Markup Language (XML) or JavaScript Object Notation (JSON) format. An example might be a patient communication device API that can be added to a hospital or medical provider website to facilitate medical services and automatically order emergency medical care.

In one aspect of the technology, the practitioner communication device (e.g., mobile phone, tablet, etc.) contains a repository (or access to a repository via a local or cloud-based server) of information related to the patient including, but not limited to, the patient's medical history and other useful information for diagnosing and prescribing patient care such as the patient's historical use of the programmable inhaler 300. According to one aspect of the technology, a cloud server is configured to provide patient data processing services to a variety of clients, such as physicians from medical institutes, sole practitioners, patients, medical researchers, regulating bodies, etc. A cloud server has the capability of processing data from one or more devices 300, practitioner communication devices, or patient communication devices (discussed below) to allow multiple participants to view and process data from device 300 and/or regulate operation of the device 300. Different participants may participate in different stages of device operation dependent upon the privileges associated with their roles (e.g., doctors, patients, emergency responders, etc.). Different participants may be limited to access only a portion of information relating to the device 300 without compromising the privacy of the patients. According to one aspect, a cloud-based medical data processing system includes a data gateway manager to automatically and/or manually transfer medical data to/from data providers such as medical institutes or emergency responders. Such data gateway management may be performed based on a set of rules or policies, which may be configured by an administrator or authorized personnel. In one aspect, in response to updates to medical data retrieved from device 300 or data processing operations performed at the cloud, the data gateway manager is configured to transmit over a network (e.g., Internet or intranet) the updated data or data representing the difference between the updated data and the original data to a data provider. Similarly, the data gateway manager may be configured to transfer any new data from the data provider and store them in a data store of the cloud-based system. In addition, the data gateway manager may further transfer data amongst multiple data providers that are associated with the same entity (e.g., multiple medical practitioners providing services to the same patient). The gateway manager may comprise a router, a computer, software or any combination of these components.

The practitioner device and practitioner API interface with specific patient history and patient data associated with the patient's use of the device 300. In one aspect of the technology, the practitioner API retrieves data about the specific patient to calculate a percentage of predicted peak expiratory flow. The peak expiratory flow (PEF), also called peak expiratory flow rate (PEFR) is a person's maximum speed of expiration, as measured with a peak flow meter. PEFR is a conventional measure of the airflow through the bronchi and thus the degree of obstruction in the airways of the patient. Peak flow readings are higher when patients are in a normal state, and lower when the airways are constricted. From changes in recorded values, patients and doctors may determine lung functionality, the severity of symptoms, and treatment. The normal expected value of PEFR depends on the patient's sex, age, and height, among other variables pulled from the patient's history compared to a repository of information related to a normal expected value. In one aspect of the technology, when a user wishes to conduct a PEFR test, the user first removes the mouthpiece 301 from device 300 and sets the device 300 to a PEFR mode. This may be done either through the patient API or directly on the device 300 by means of a switch. In another aspect, a contact sensor between the mouthpiece 301 and housing 302 automatically sets the device 300 to PEFR mode when the mouthpiece 301 is removed. The device 300 is configured be in “medicine dispensing mode” when the mouthpiece 301 is connected to the housing 302. In this aspect, a separate port (e.g., a one-way valve) on the bottom of the housing 302 permits expiratory air from the patient to leave the housing 302. In another aspect of the technology, when the device 300 is in PEFR mode, the mouthpiece 301 remains coupled to the housing 302 when conducting the PEFR test. However, the PEEP valve associated with the mouthpiece 301 is removed, creating a port on the mouthpiece for expiratory air from the patient. In still another aspect of the technology, the PEFR test may be conducted with the device 300 in its completely assembled stated.

In one aspect of the technology, the device 300 is programmed to respond to a plurality of different commands from the PCB 308 (or other device circuitry) based on the patient's use of the device 300 and how the measured PEFR compares to a percentage of what the predicted or desired PEFR of the patient should be. For example, in one aspect, through the practitioner API, the device 300 may be programmed to provide a plurality of different operational messages to the patient based on the patient's measured PEFR. In this example, if the patient feels discomfort and wishes to use the device 300 to relieve unwanted symptoms, the patient may blow on the device 300. This might also be done on a scheduled basis. If the measured PEFR is greater than 80% of that patients expected PEFR as programmed by the physician a message of GOOD, green light and/or audible indicator is performed. The GOOD message may appear on the display 310, for example. If the measured PEFR ranges between 60% and 80%, a message of BEHIND TRY AGAIN, yellow light, and/or audible indicator may be performed. If the measured PEFR ranges between 40% and 60%, a message of CONTACT PHYSICIAN, orange light, and/or audible indicator may appear. If the measured PEFR is below 40% a message of CALL 911 and a red light may appear. While exemplary ranges are provided herein, the ranges of PEFR triggered commands are examples only and should not be considered limiting to the technology in any way.

Forced expiratory volume in the first second of exhalation (FEV1) is also derived from the flow sensor 309 and calculated by the logic card and subsequently read out on the display 310 while being transferred to the blue tooth. In one aspect of the technology, forced expiratory volume or FEV1 is calculated based on changes to pressure detected by sensor 309. For example, Bernoulli's Principle is embodied by the following equation:

P₁+pgh+½pv₁²−P₂+pgh+½v₂²

where P is pressure, h is elevation, ρ is density, v is velocity and g is the gravitational acceleration. Assuming v2 is 0 and is the velocity at the sensor 309, h1 and h2 are equal, velocity is equal to mass flow rate Q divided by an area A (e.g., the cross-sectional area of the mouthpiece or other chamber, for example) as follows:

$$V = \frac{Q}{A}$$

$$Δ P = \frac{1}{2} {pv}_{1}^{2}$$

$$Δ P = \frac{1}{2} {p ( \frac{Q}{A} )}^{2}$$

Pressure change can therefore be used to calculate the flow rate and particularly the forced expiratory volume occurring during the first second (or first few seconds) of exhalation. Other mathematical models may be used to calculate FEV1 based on the use of any number of different pressure sensor device or pressure calculation techniques.

In another aspect of the technology, the device 300 is capable of also coupling with a blue tooth (or other data communication link) enabled device 320 belonging to the patient (i.e., the patient communication device). The patient communication device records data measured from device 300 and stores the information locally on the patient communication device and/or on a remote repository such as a cloud-based server. The patient communication device is equipped with a patient API that allows the patient to track his/her collected data from sensor 309 and track any improvement or degradation in PEFR or FEV1, for example. In addition, the API of the patient communication device may be programmed to permit the patient, to create an automated command structure in an emergency situation. For example, in one aspect of the technology, referencing the operation protocols (GOOD, TRY AGAIN, etc.) noted above, the patient communication device may be programmed to automatically call 911 if the measured PEFR (or other measured medical data such as FEV1) is below 20% (or some other predetermined metric) for one, or more than one attempt at self-administration of the medication through device 300. In another aspect, the patient communication device may be programmed to send a text message or place an automated call to a medical professional when the measured PEFR (or other measured data point) is within a predetermined range. In still another aspect, all, or substantially all, of the measured data collected from use of the device 300 is uploaded to the patient communication device, the medical practitioner communication device, and/or the cloud to be used to treat the patient and understand patient needs. In one aspect, the patient communication device and associated API does not have permission to modify the prescribed settings of the device 300. Rather, the patient communication device (and/or patient API) is configured primarily to gather and transmit data related to the use and operation of the device 300 and communicate with medical practitioners and/or emergency services based on the patient's measured medical status. The command structure and prescribed treatment based on device operational parameters may only be modified through the practitioner API.

The device 300, patient communication device (and/or related API), and practitioner communication device (and/or related API) form a system that may be used as part of a server or a client. The use of a cloud-based system is not intended to limit any specific architecture or manner of interconnecting the system components. It will also be appreciated that network computers, handheld computers, cell phones and other data processing systems which have fewer components or perhaps more components may also be used with the present technology. The present technology may also utilize a non-volatile memory which is remote from the device 300; such as, a network storage device which is coupled to the data processing system through a network interface such as a modem or Ethernet interface. A bus may include one or more buses connected to each other through various bridges, controllers, and/or adapters, as is well-known in the art. In one embodiment, the I/O controller includes a USB (Universal Serial Bus) adapter for controlling USB peripherals. Alternatively, I/O controller may include an IEEE adapter, also known as FireWire adapter, for controlling FireWire devices.

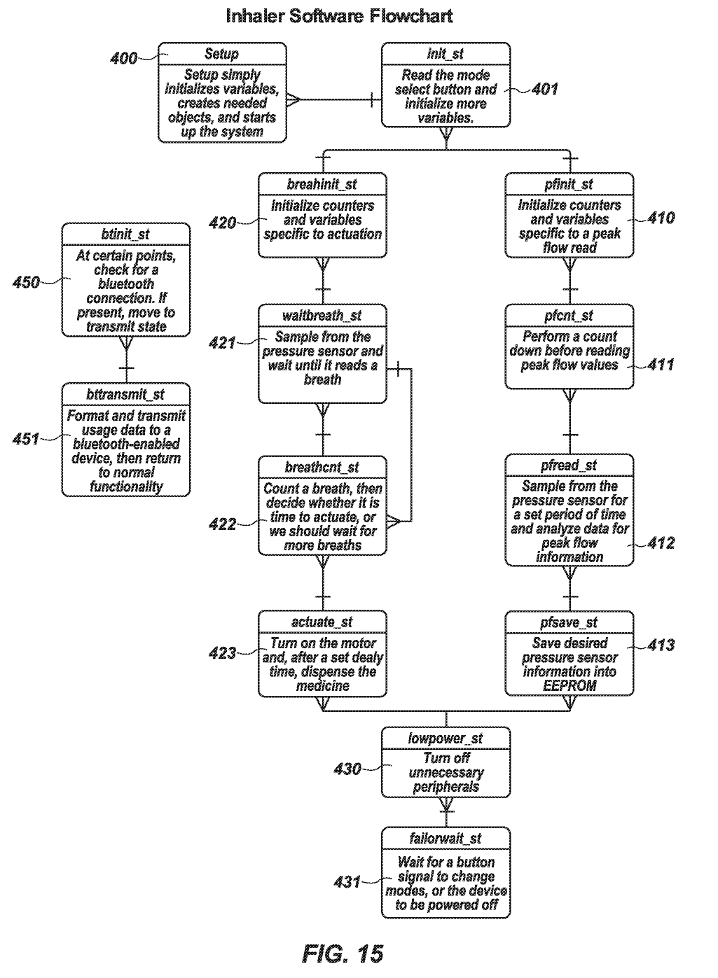

With reference to FIG. 15, in one aspect of the technology, the device 300 comprises a system that is software and/or hardware based that starts with a setup step 400. The step initializes variables, creates objects, and overall initiates the system. Step 401 determines which mode the device has been set. In one aspect, where the device has been set to sample a user's peak expiratory flow without administering medication, step 410 is initiated which initializes the system counters and variables to read a peak flow measurement. The system performs a visible countdown through the LEDs at step 411 and then samples the pressure sensor 309 at step 412 for a predetermined period of time and analyzes the data to determine peak flow information. At step 413, this information is saved to the EEPROM. In a medication administration mode, step 420 is taken which initializes the counters and variables specific to actuation of the device 300 resulting in administration of a medication. At step 421, at least one sample is taken from the pressure senor 309 until a “breath” (i.e., an increase in pressure) is detected. At step 422, the system determines if the breath is a “qualified breath” and if the number of consecutive “qualified breaths” or the number of “qualified breaths” within a predetermined period of time has been counted. If the number has been reached, at step 423 the motor 306 is actuated and medication is administered. At step 430, the system powers down unnecessary peripherals and waits for a change in mode or powers off. During periods of operation, the system is also set to check for a bluetooth (or other wireless) connection at step 450 for a remote device 320 (e.g., a practitioner communication device and/or a patient communication device). If an authorized device 320 is located, the system is set to a transmit step where at step 451 data from the device 300 is formatted and transmitted to device 320 (directly and/or through a cloud-based database).

In accordance with one aspect of the technology, the device 300 has controls for operating the medical device comprising a lock that prevents at least some functions of the inhalator from being operable through the controls. The lock granting initial access to the may be configured to perform machine-recognition of a biometric indicator of identity, such as fingerprint recognition, facial recognition. However, the lock may also be opened simply through entry of a numerical, alphabetical, alphanumeric, or related code corresponding to a specific patient. In this manner, each device 300 may be “updated” through a related patient and/or physician API (or connection to another remote database with relevant information stored therein) to correspond to the patient's specific prescription. Meaning, the number of qualified breaths or requirements to achieve a qualified breath would be automatically updated by entry of a code corresponding to a particular patient. This would permit different patients to use the same device 300 with a specific treatment program for each patient. The display 310 provides a message that displays on the display 310 requirements for a treatment procedure specific to the patient's identity. The key may include a magnetic medium storing a data key. The lock, may include a video camera and a device configured for machine-classification of a video image from the video camera. The lock may be a simple structure, for example one that prevent access by locking out the controls. The lock, however, would not prevent manual operation of the trigger.

Some portions of the preceding detailed descriptions have been presented in terms of representations of operations within a computer memory. It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as those set forth in the claims below, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices. Known techniques can be implemented using code and data stored and executed on one or more electronic devices. Such electronic devices store and communicate (internally and/or with other electronic devices over a network) code and data using computer-readable media, such as non-transitory computer-readable storage media (e.g., magnetic disks; optical disks; random access memory; read only memory; flash memory devices; phase-change memory) and transitory computer-readable transmission media (e.g., electrical, optical, acoustical or other form of propagated signals—such as carrier waves, infrared signals, digital signals). The processes or methods described may be performed by processing logic that comprises hardware (e.g. circuitry, dedicated logic, etc.), firmware, software (e.g., embodied on a non-transitory computer readable medium), or a combination of both. Although the processes or methods are described above in terms of some sequential operations, it should be appreciated that some of the operations described may be performed in a different order. Moreover, some operations may be performed in parallel rather than sequentially.

1. A system for setting a medically prescribed treatment regime on a hand held inhalator device, comprising:
a medical practitioner communication device untethered from the hand held inhalator, said medical practitioner communication device comprising a radio transmitter/receiver configured to receive data from the hand held inhalator device and transmit data to the hand held inhalator device;
the hand held inhalator device having (i) a transmitter/receiver configured to receive data from the medical practitioner device and send data to the medical practitioner device; (ii) a valve disposed about a fluid outlet, the valve configured to open when a portion of the inhalator device is subjected to a first threshold level of positive pressure; and (iii) a trigger configured to dispense medication from a medication source into a chamber of the inhalator device after a second threshold level of positive pressure is achieved within the chamber and maintained for a predetermined period of time; and
wherein the predetermined period of time and the second threshold level of positive pressure is configurable by data received from the medical practitioner communication device.

2. The system of claim 1, wherein the medical practitioner communication device comprises an application programming interface that has been uploaded to the medical practitioner device, said application programming interface being configured to permit a medical practitioner to configure the predetermined period of time and the second threshold level of positive pressure required to actuate the trigger of the hand held inhalator device.

3. The system of claim 2, wherein the medical practitioner communication device is configured to communicate with the hand held inhalator device directly, or indirectly through a remote database.

4. The system of claim 1, further comprising a display disposed about a lateral side of the hand held inhalator device.

5. The system of claim 4, wherein the display is configured to provide instructions to the user of the hand held inhalator device corresponding to the user's expected expiratory flow rate.

6. The system of claim 1, wherein the inhalator device is further configured to receive data from a patient communication device and transmit data to the patient communicator device.

7. The system of claim 6, wherein the patient communication device is configured to communicate with the hand held inhalator device directly, or indirectly through a remote database.

8. The system of claim 7, wherein the predetermined period of time and the second threshold level of positive pressure is configurable by data received from the patient communication device.

9. The system of claim 6, wherein the patient communication device is programmed to call for emergency medical assistance if the patient's peak expiratory flow value or forced expiratory volume measured by the inhalator device is below a threshold value.

10. A system for operating a medically prescribed hand held inhalator device, comprising:
the hand held inhalator device having (i) a transmitter/receiver configured to receive data from the medical practitioner device and send data to the medical practitioner device; (ii) a valve disposed about a fluid outlet, the valve configured to open when a portion of the inhalator device is subjected to a first threshold level of positive pressure; and (iii) a trigger configured to dispense medication from a medication source into a chamber of the inhalator device after a second threshold level of positive pressure is achieved within the chamber and maintained for a predetermined period of time; and
a medical practitioner application programming interface configurable for use on a medical practitioner communication device, said medical practitioner communication device comprising a radio transmitter/receiver configured to receive data from the hand held inhalator device and transmit data to the hand held inhalator device through the medical practitioner application programming interface;
wherein the predetermined period of time and the second threshold level of positive pressure of the hand held inhalator device is configurable for a specific patient prescription by data received from the medical practitioner communication device through the medical practitioner programming interface.

11. The system of claim 10, further comprising a patient device application programming interface configurable for use on a patient communication device, said patient communication device comprising a radio transmitter/receiver configured to receive data form the hand held inhalator device and transmit data to the hand held inhalator device through the patient device application programming interface.

12. The system of claim, wherein the hand held inhalator device comprises a display coupled to a memory storage and a processor, the display configured to provide operational messages based on the patients' measured peak expiratory flow rate.

13. The system of claim 10, wherein the inhalator device comprises a locking mechanism configured to modify operational characteristics of the inhalator device specific to a patient prescription when the locking mechanism is unlocked.

14. The system of claim 10, wherein the inhalator device comprises a sensor disposed within the chamber, said sensor configured to measure the pressure and temperature within the chamber.

15. The system of claim 10, further comprising a remote database containing a medical history of the patient, the patient's recorded peak expiratory flow rate measurements from the inhalator device, and the patient's currently prescribed inhalator treatment program, said program comprising at least the number of qualifying breaths the patient must achieve before the trigger of the inhalator device is actuated.

16. A method of operating a programmable inhalator device, comprising:
obtaining a hand held inhalator device, said inhalator device comprising (i) a transmitter/receiver configured to receive data from a medical practitioner device and send data to the medical practitioner device; (ii) a valve configured to open when a portion of the inhalator device is subjected to a first threshold level of positive pressure; (iii) a sensor located within a chamber of the inhalator device, said sensor configured to detect pressure within the chamber; and (iii) a trigger configured to dispense medication from a medication source into a chamber of the inhalator device after a predetermined number of qualifying breaths have been detected by the hand held inhalator device, said qualifying breath comprising a second threshold level of positive pressure detected within the chamber and maintained for a predetermined period of time;
setting the second threshold level of positive pressure and the predetermined period of time required for the inhalator device to register a qualified breath through an application programming interface, said predetermined period of time of said second threshold level of positive pressure corresponding to a specific patient prescription; and
setting the number of qualifying breaths to be registered by the device before actuation of the trigger through the application programming interface, said number of qualifying breaths corresponding to a specific patient prescription.

17. The method of claim 16, wherein a physician sets the predetermined period of time of said second threshold level of positive pressure and the number of qualifying breaths required to actuate the trigger through an application programming interface located on the physician's communication device.

18. The method of claim 16, wherein a patient sets the predetermined period of time of said second threshold level of positive pressure and the number of qualifying breaths required to actuate the trigger through an application programming interface located on the patient's communication device.

19. The method of claim 18, wherein the patient can set the predetermined period of time of said second threshold level of positive pressure and the number of qualifying breaths required to actuate the trigger through an application programming interface located on the patient's communication device only after inputting an authorization code received from the physician.

20. The method of claim 16, comprising the step of administering medication to the patient after a prescribed number of prescribed qualifying breaths has been detected by the inhalator device.
